(19) United States
(12) Patent Application Publication
(10) Pub. No.: US 2021/0319560 A1
(43) Pub. Date: Oct. 14, 2021

(54) IMAGE PROCESSING METHOD AND APPARATUS, AND STORAGE MEDIUM

(71) Applicant: BEIJING SENSETIME TECHNOLOGY DEVELOPMENT CO., LTD., Beijing (CN)

(72) Inventors: Qing XIA, Beijing (CN); Ning HUANG, Beijing (CN)

(73) Assignee: BEIJING SENSETIME TECHNOLOGY DEVELOPMENT CO., LTD., Beijing (CN)

(21) Appl. No.: 17/356,398

(22) Filed: Jun. 23, 2021

Related U.S. Application Data
(63) Continuation of application No. PCT/CN2019/107844, filed on Sep. 25, 2019.

(30) Foreign Application Priority Data
Apr. 1, 2019 (CN) 201910258038.1

(51) Int. Cl. G06T 7/11; G06K 9/62; G06T 7/00; G06T 3/40; A61B 5/00

(52) U.S. Cl. CPC G06T 7/11; G06K 9/6232; G06T 7/0012; G06T 3/4046; A61B 2576/023; G06T 2207/20084; G06T 2207/20081; G06T 2207/30048; A61B 5/7267

(57) ABSTRACT

The present disclosure relates to an image processing method and apparatus, and a storage medium. The method includes: performing step-by-step convolution processing on an image to be processed to obtain a convolution result (S11); obtaining a positioning result through positioning processing according to the convolution result (S12); performing step-by-step deconvolution processing on the positioning result to obtain a deconvolution result (S13); and performing segmentation processing on the deconvolution result to segment a target object from the image to be processed (S14). Embodiments of the present disclosure implement target object positioning and segmentation at the same time in a process of image processing, and the image processing precision is improved while the speed of image processing is guaranteed.

### CROSS-REFERENCE TO RELATED APPLICATIONS

The present application is a bypass continuation of and claims priority under 35 U.S.C. § 111(a) to PCT Application. No. PCT/CN2019/107844, filed on Sep. 25, 2019, which claims priority to Chinese Patent Application No. 201910258038.1, filed with the Chinese Patent Office on Apr. 1, 2019 and entitled “IMAGE PROCESSING METHOD AND APPARATUS, ELECTRONIC DEVICE, AND STORAGE MEDIUM”, each of which is incorporated herein by reference in its entirety.

### TECHNICAL FIELD

The present disclosure relates to the technical field of image processing, and in particular, to an image processing method and apparatus, an electronic device, and a storage medium.

### BACKGROUND

In technical field of images, segmenting areas of interest or target areas is the basis of image analysis and target recognition. For example, boundaries between one or more organs or lesions are clearly recognized by means of segmentation in medical images. Accurately segmenting a three-dimensional medical image is critical to many clinical applications.

### SUMMARY

The present disclosure provides technical solutions of image processing.

According to one aspect of the present disclosure, provided is an image processing method, including: performing step-by-step convolution processing on an image to be processed to obtain a convolution result; obtaining a positioning result through positioning processing according to the convolution result; performing step-by-step deconvolution processing on the positioning result to obtain a deconvolution result; and performing segmentation processing on the deconvolution result to segment a target object from the image to be processed.

In one possible implementation mode, performing step-by-step convolution processing on the image to be processed to obtain the convolution result includes: performing step-by-step convolution processing on the image to be processed to obtain at least one feature map having gradually decreasing resolution as the convolution result.

In one possible implementation mode, performing step-by-step convolution processing on the image to be processed to obtain the at least one feature map having the gradually decreasing resolution as the convolution result includes: performing convolution processing on the image to be processed, where an obtained feature map serves as a feature map to be convolved; the resolution of the feature map to be convolved does not reach a first threshold, performing convolution processing on the feature map to be convolved and taking the obtained result as a feature map to be convolved again; and when the resolution of the feature map to be convolved reaches the first threshold, taking all the feature maps having the gradually decreasing resolution as the convolution result.

In one possible implementation mode, obtaining the positioning result through positioning processing according to the convolution result includes: performing segmentation processing according to the convolution result to obtain a segmentation result; and performing positioning processing on the convolution result according to the segmentation result to obtain the positioning result.

In one possible implementation mode, performing segmentation processing according to the convolution result to obtain the segmentation result includes: performing segmentation processing on the feature map having the lowest resolution in the convolution result to obtain the segmentation result.

In one possible implementation mode, performing positioning processing on the convolution result according to the segmentation result to obtain the positioning result includes: determining corresponding position information of the target object in the convolution result according to the segmentation result; and performing positioning processing on the convolution result according to the position information to obtain the positioning result.

In one possible implementation mode, determining the corresponding position information of the target object in the convolution result according to the segmentation result includes: reading an coordinate position of the segmentation result; and taking the coordinate position as an area center, respectively determining, in the convolution result, an area position capable of fully covering the target object in the feature map at each resolution as the corresponding position information of the target object in the convolution result.

In one possible implementation mode, performing positioning processing on the convolution result according to the position information to obtain the positioning result includes: respectively performing cropping processing on the feature map at each resolution in the convolution result according to the position information to obtain the positioning result.

In one possible implementation mode, performing step-by-step deconvolution processing on the positioning result to obtain the deconvolution result includes: taking the feature map having the lowest resolution in all the feature maps included in the positioning result as a feature map to be deconvolved; when the resolution of the feature map to be deconvolved does not reach a second threshold, performing deconvolution processing on the feature map to be deconvolved to obtain a deconvolution processing result; determining the next feature map of the feature map to be deconvolved in the positioning result according to a gradually increasing resolution order; fusing the deconvolution processing result and the next feature map, and taking the fusing result as a feature map to be deconvolved again; and when the resolution of the feature map to be deconvolved reaches the second threshold, taking the feature map to be deconvolved as the deconvolution result.

In one possible implementation mode, the segmentation processing includes: performing softmax regression on an object to be segmented to obtain a regression result; and performing maximum value comparison on the regression result to complete the segmentation processing on the object to be segmented.

In one possible implementation mode, the method is implemented by a neural network, and the neural network includes a first segmentation sub-network and a second segmentation sub-network, where the first segmentation sub-network is configured to perform step-by-step convolution processing and segmentation processing on the image to be processed, and the second segmentation sub-network is configured to perform step-by-step deconvolution processing and segmentation processing on the positioning result.

In one possible implementation mode, a training process for the neural network includes: training the first segmentation sub-network according to a preset training set; and training the second segmentation sub-network according to the preset training set and the trained first segmentation sub -network.

In one possible implementation mode, before performing step-by-step convolution processing on the image to be processed to obtain the convolution result, the method further includes: adjusting the image to be processed to preset resolution.

In one possible implementation mode, the image to be processed is a three-dimensional medical image.

According to one aspect of the present disclosure, provided is an image processing apparatus, including: a convolution module, configured to perform step-by-step convolution processing on an image to be processed to obtain a convolution result; a positioning module, configured to obtain a positioning result through positioning processing according to the convolution result; a deconvolution module, configured to perform step-by-step deconvolution processing on the positioning result to obtain a deconvolution result; and a target object obtaining module, configured to perform segmentation processing on the deconvolution result to segment a target object from the image to be processed.

In one possible implementation mode, the convolution module is configured to: perform step-by-step convolution processing on the image to be processed to obtain at least one feature map having gradually decreasing resolution as the convolution result.

In one possible implementation mode, the convolution module is further configured to: perform convolution processing on the image to be processed, where an obtained feature map serves as a feature map to be convolved; the resolution of the feature map to be convolved does not reach a first threshold, perform convolution processing on the feature map to be convolved and take the obtained result as a feature map to be convolved again; and when the resolution of the feature map to be convolved reaches the first threshold, take all the feature maps having the gradually decreasing resolution as the convolution result.

In one possible implementation mode, the positioning module includes: a segmentation sub-module, configured to perform segmentation processing according to the convolution result to obtain a segmentation result; and a positioning sub-module, configured to perform positioning processing on the convolution result according to the segmentation result to obtain the positioning result.

In one possible implementation mode, the segmentation sub-module is configured to: perform segmentation processing on the feature map having the lowest resolution in the convolution result to obtain the segmentation result.

In one possible implementation mode, the positioning sub-module is configured to: determine corresponding position information of the target object in the convolution result according to the segmentation result; and perform positioning processing on the convolution result according to the position information to obtain the positioning result.

In one possible implementation mode, the positioning sub-module is further configured to: read a coordinate position of the segmentation result; and taking the coordinate position as an area center, respectively determine, in the convolution result, an area position capable of fully covering the target object in the feature map at each resolution as the corresponding position information of the target object in the convolution result.

In one possible implementation mode, the positioning sub-module is further configured to: respectively perform cropping processing on the feature map at each resolution in the convolution result according to the position information to obtain the positioning result.

In one possible implementation mode, the deconvolution module is configured to: take the feature map having the lowest resolution in all the feature maps included in the positioning result as a feature map to be deconvolved; when the resolution of the feature map to be deconvolved does not reach a second threshold, perform deconvolution processing on the feature map to be deconvolved to obtain a deconvolution processing result; determine the next feature map of the feature map to be deconvolved in the positioning result according to a gradually increasing resolution order; fuse the deconvolution processing result and the next feature map, and take the fusing result as a feature map to be deconvolved again; and when the resolution of the feature map to be deconvolved reaches the second threshold, take the feature map to be deconvolved as the deconvolution result.

In one possible implementation mode, the segmentation processing includes: performing softmax regression on an object to be segmented to obtain a regression result; and performing maximum value comparison on the regression result to complete the segmentation processing on the object to be segmented.

In one possible implementation mode, the apparatus is implemented by a neural network, and the neural network includes a first segmentation sub-network and a second segmentation sub-network, where the first segmentation sub-network is configured to perform step-by-step convolution processing and segmentation processing on the image to be processed, and the second segmentation sub-network is configured to perform step-by-step deconvolution processing and segmentation processing on the positioning result.

In one possible implementation mode, the apparatus further includes a training module, configured to: train the first segmentation sub-network according to a preset training set; and train the second segmentation sub-network according to the preset training set and the trained first segmentation sub-network.

In one possible implementation mode, before the convolution module, the apparatus further includes a resolution adjusting module, configured to: adjust the image to be processed to preset resolution.

In one possible implementation mode, the image to be processed is a three-dimensional medical image.

According to one aspect of the present disclosure, provided is an electronic device, including: a processor; and a memory configured to store processor executable instructions, where the processor is configured to: execute the foregoing image processing method.

According to one aspect of the present disclosure, provided is a computer-readable storage medium having computer program instructions stored thereon, where when the computer program instructions are executed by a processor, the foregoing image processing method is implemented.

In embodiments of the present disclosure, by performing step-by-step convolution processing and segmentation processing on an image to be processed to obtain a segmentation result, obtaining a positioning result based on the segmentation result, and then performing step-by-step deconvolution processing on the positioning result and then segmentation processing, a target object is segmented from the image to be processed. According to the process above, target object positioning and segmentation are implemented at the same time in a process of image processing, and the image processing precision is improved while the speed of image processing is guaranteed.

It should be understood that the foregoing general descriptions and the following detailed descriptions are merely exemplary and explanatory, but are not intended to limit the present disclosure. Exemplary embodiments are described in detail below according to the following reference accompanying drawings, and other features and aspects of the present disclosure become clear.

### BRIEF DESCRIPTION OF THE DRAWINGS

The accompanying drawings here are incorporated into the specification and constitute a part of the specification. These accompanying drawings show embodiments that conform to the present disclosure, and are intended to describe the technical solutions in the present disclosure together with the specification.

FIG. 1 is a flowchart illustrating an image processing method according to one embodiment of the present disclosure.

FIG. 2 is a flowchart illustrating an image processing method according to one embodiment of the present disclosure.

FIG. 3 is a flowchart illustrating an image processing method according to one embodiment of the present disclosure.

FIG. 4 is a flowchart illustrating an image processing method according to one embodiment of the present disclosure.

FIG. 5 is a flowchart illustrating an image processing method according to one embodiment of the present disclosure.

FIG. 6 is a flowchart illustrating an image processing method according to one embodiment of the present disclosure.

FIG. 7 is a flowchart illustrating an image processing method according to one embodiment of the present disclosure.

FIG. 8 is a schematic diagram of one application example of the present disclosure.

FIG. 9 is a block diagram illustrating an image processing apparatus according to one embodiment of the present disclosure.

FIG. 10 is a block diagram of an electronic device according to embodiments of the present disclosure.

FIG. 11 is a block diagram of an electronic device according to embodiments of the present disclosure.

### DETAILED DESCRIPTION

The following describes various exemplary embodiments, features, and aspects of the present disclosure in detail with reference to the accompanying drawings. Same reference numerals in the accompanying drawings represent elements with same or similar functions. Although various aspects of the embodiments are illustrated in the accompanying drawings, the accompanying drawings are not necessarily drawn in proportion unless otherwise specified.

The special term “exemplary” here refers to “being used as an example, an embodiment, or an illustration”. Any embodiment described as “exemplary” here should not be explained as being more superior or better than other embodiments.

The term “and/or” herein describes only an association relationship describing associated objects and represents that three relationships may exist. For example, A and/or B may represent the following three cases: only A exists, both A and B exist, and only B exists. In addition, the term “at least one” herein indicates any one of multiple listed items or any combination of at least two of multiple listed items. For example, including at least one of A, B, or C may indicate including any one or more elements selected from a set consisting of A, B, and C.

In addition, for better illustration of the present disclosure, various specific details are given in the following specific implementations. A person skilled in the art should understand that the present disclosure may also be implemented without the specific details. In some instances, methods, means, elements, and circuits well known to a person skilled in the art are not described in detail so as to highlight the subject matter of the present disclosure.

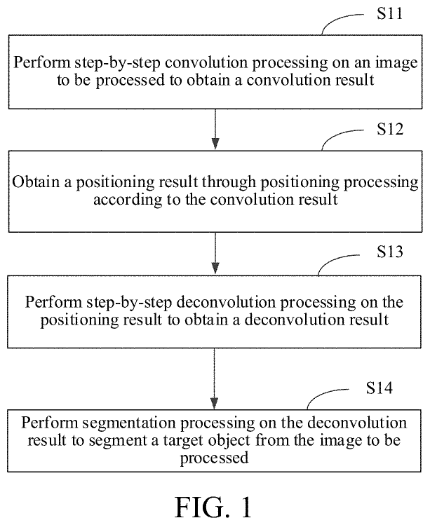

FIG. 1 is a flowchart illustrating an image processing method according to one embodiment of the present disclosure. The method is applicable to an image processing apparatus, which may be a terminal device, a server, other processing device, or the like. The terminal device may be a User Equipment (UE), a mobile device, a user terminal, a terminal, a cellular phone, a cordless telephone, a Personal Digital Assistant (PDA), a handheld device, a computer device, a vehicle-mounted device, a wearable device, or the like.

In some possible implementation modes, the image processing method may be implemented by invoking, by a processor, computer readable instructions stored in a memory.

As shown in FIG. 1, the image processing method includes the following steps.

At step S11, step-by-step convolution processing is performed on an image to be processed to obtain a convolution result.

At step S12, a positioning result is obtained through positioning processing according to the convolution result.

At S13, step-by-step deconvolution processing is performed on the positioning result to obtain a deconvolution result.

At step S14, segmentation processing is performed on the deconvolution result to segment a target object from the image to be processed.

According to the image processing method of the embodiments of the present disclosure, by means of step-by-step convolution processing and segmentation processing, preliminary segmentation is performed on a target object in an image to be processed, so that a positioning result reflecting a basic distribution position of the target object in the image to be processed is obtained. Based on the positioning result, high-precision segmentation is further performed on the target object in the image to be processed by means of step-by-step deconvolution processing and segmentation processing, based on this process, segmentation of the target object is implemented on the basis of the positioning result, and compared with direct target segmentation on the image to be processed, the precision of image processing is effectively improved. Moreover, the method can be used in one image processing process, where an image is subjected to target positioning and segmentation, and because analysis is made combining target positioning and segmentation processes of the image, time consumption of image processing is reduced, and storage consumption which may exist in the image processing process is also reduced.

The image processing method of the embodiments of the present disclosure is applied to processing of three-dimensional medical images, for example, for recognizing a target area in the medical image, where the target area may be an organ, a lesion, a tissue, or the like. In one possible implementation mode, the image to be processed is a three-dimensional medical image of the heart organ, that is, the image processing method of the embodiments of the present disclosure may be applied to a treatment process for the heart disease. In one example, the image processing method may be applied to a treatment process for atrial fibrillation. By precisely segmenting an image of the atrium, the cause of the atrial fibrillation is understood and analyzed, then a surgical ablation therapeutic plan targeting the atrial fibrillation is formulated, and the therapeutic effect for the atrial fibrillation is improved.

It should be noted that the image processing method of the embodiments of the present disclosure is not limited to application in three-dimensional medical image processing, and may be applied to any image processing, which is not limited by the present disclosure.

In one possible implementation mode, the image to be processed may include a plurality of images, and one or more three-dimensional organs are recognized from the plurality of images.

The implementation mode of step S11 is not limited, and any mode capable of obtaining a feature map for segmentation processing may be taken as the implementation mode of step S11. In one possible implementation mode, step S11 includes: performing step-by-step convolution processing on the image to be processed to obtain at least one feature map having gradually decreasing resolution as the convolution result.

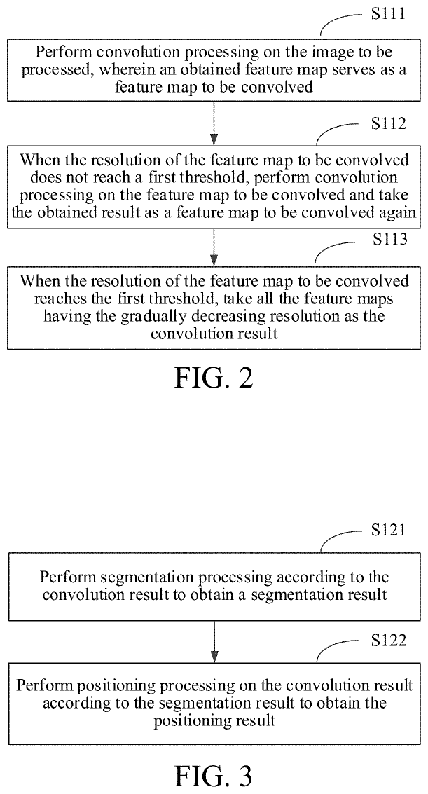

Regarding how the at least one feature map having gradually decreasing resolution is obtained by means of step-by-step convolution processing, the specific processing process is likewise not limited. FIG. 2 is a flowchart illustrating an image processing method according to one embodiment of the present disclosure. As shown in FIG. 2, in one possible implementation mode, performing step-by-step convolution processing on the image to be processed to obtain the at least one feature map having the gradually decreasing resolution as the convolution result includes the following steps.

At step S111, convolution processing is performed on the image to be processed, where an obtained feature map serves as a feature map to be convolved.

At step S112, the resolution of the feature map to be convolved does not reach a first threshold, convolution processing is performed on the feature map to be convolved and the obtained result is taken as a feature map to be convolved again.

At step S113, when the resolution of the feature map to be convolved reaches the first threshold, all the feature maps having the gradually decreasing resolution are taken as the convolution result.

It can be seen from the steps above that in the embodiments of the present disclosure, by performing convolution processing on the image to be processed, a feature map under initial resolution is obtained, and then by performing another convolution processing on the feature map under the initial resolution, a feature map under the next resolution is obtained, and so forth, so that a series of feature maps having gradually decreasing resolution are obtained, and the feature maps are taken as the convolution result for subsequence steps. The number of iterations in this process is not limited. The process stops when the obtain feature map having the lowest resolution reaches the first threshold. The first threshold may be set according to needs and actual conditions, and the specific value is not limited herein. Because the first threshold is not defined, the number of feature maps and the resolution of each feature map included in the obtained convolution result are not limited, and may be specifically selected according to actual conditions.

In one possible implementation mode, the convolution processing process and implementation mode are not limited. In one example, the convolution processing process may include performing one or more of convolution, pooling, batch normalization, or Parametric Rectified Linear Unit (PReLU) on a to-be-process object. In one example, it may be implemented by using an encoder structure in a 3D U-Net full convolutional neural network. In one example, it may also be implemented by using an encoder structure in a V-Net full convolutional neural network. The specific mode of the convolution processing is not limited in the present disclosure.

According to the convolution result, there is a plurality of implementation modes for the process of obtaining a positioning result by means of positioning processing. FIG. 3 is a flowchart illustrating an image processing method according to one embodiment of the present disclosure. As shown in FIG. 3, in one implementation mode, step S12 includes the following steps.

At step S121, segmentation processing is performed according to the convolution result to obtain a segmentation result.

At step S122, positioning processing is performed on the convolution result according to the segmentation result to obtain the positioning result.

The process of step S121 is likewise not limited. It can be known from the embodiments of the disclosure above, the convolution result may include a plurality of feature maps, and therefore, the segmentation result is obtained by performing segmentation processing on which feature map in the convolution result may be determined according to actual conditions. In one possible implementation mode, steps S121 includes: performing segmentation processing on the feature map having the lowest resolution in the convolution result to obtain the segmentation result.

The processing mode of the segmentation processing is not limited, and any mode capable of segmenting a target from a feature map may be taken as the segmentation processing method in examples of the present disclosure.

In one possible implementation mode, the segmentation processing may be implementing image segmentation by means of a softmax layer, and the specific process includes: performing softmax regression on an object to be segmented to obtain a regression result; and performing maximum value comparison on the regression result to complete the segmentation processing on the object to be segmented. In one example, the specific process of performing maximum value comparison on the regression result to complete the segmentation processing on the object to be segmented is: the form of the regression result is output data having the same resolution as the object to be segmented, the output data has one-to-one correspondence to the pixel positions of the object to be segmented, the output data includes a probability value at each corresponding pixel position for representing the probability of the object to be segmented at the pixel position being the segmentation target, the maximum value comparison is performed based on the probabilities in the output data, so that whether each pixel position is the segmentation target position is determined, and then an operation of extracting a segmentation target from the object to be segmented is implemented. The specific mode of maximum value comparison is not limited, may be set as that the pixel position represented by a greater probability corresponds to the segmentation target, or may be set as that the pixel position represented by a smaller probability corresponds to the segmentation target. It can be set according to actual conditions, which is not limited herein. It can be known from the embodiments that in one example, the process for obtaining the segmentation result is: enabling the feature map having the lowest resolution in the convolution result to pass through a softmax layer, and performing maximum value comparison on the obtained result to obtain the segmentation result.

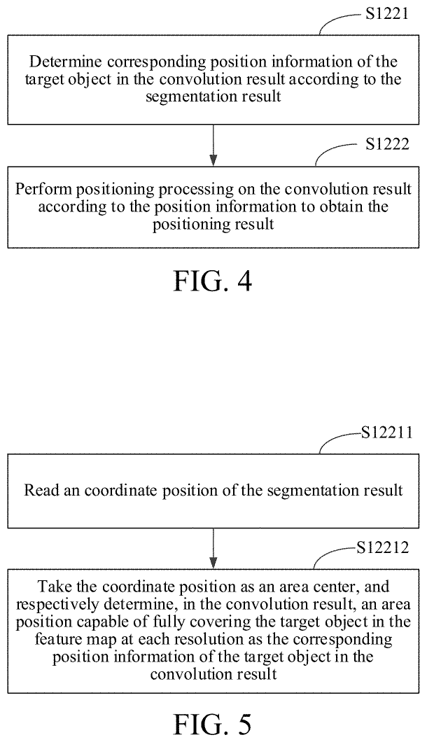

Based on the segmentation result, the positioning result is obtained by performing positioning processing on the convolution result by using step S122. The implementation mode of step S122 is not limited. FIG. 4 is a flowchart illustrating an image processing method according to one embodiment of the present disclosure. As shown in FIG. 4, in one possible implementation mode, step S122 includes the following steps.

At step S1221, corresponding position information of the target object in the convolution result is determined according to the segmentation result.

At step S122, positioning processing is performed on the convolution result according to the position information to obtain the positioning result.

The position information is information capable of indicating the position where the target object is located in the feature maps in the convolution result, and the specific representation form is not limited. In one example, the position information may be in the form of a position coordinate set. In one example, the position information may be in the form of coordinates and areas. The representation form of the position information may be flexibly selected according to actual conditions. Because the representation form of the position information is not limited, the specific process of step S1221 is flexibly determined along with the representation form of the position information. FIG. 5 is a flowchart illustrating an image processing method according to one embodiment of the present disclosure. As shown in FIG. 5, in one possible implementation mode, step S1221 includes the following steps.

At step S12211, a coordinate position of the segmentation result is read.

At step S12212, taking the coordinate position as an area center, in the convolution result, an area position capable of fully covering the target object in the feature map at each resolution is respectively determined as the corresponding position information of the target object in the convolution result.

The coordinate position of the segmentation result read in step S1221 may be any coordinates representing the position of the segmentation result. In one example, the coordinates may be coordinates of a certain fixed position on the segmentation result. In one example, the coordinates may be coordinates of certain fixed positions on the segmentation result. In one example, the coordinates may be coordinates of the center of gravity position of the segmentation result. Based on the read coordinate position, the target object is positioned at a corresponding position under each feature map in the convolution result through step S12212, and then the area position fully covering the target object is obtained. The representation form of the area position is likewise not limited. In one example, the representation form of the area position may be a coordinate set of all vertices of the area. In one example, the representation form of the area position may be a set of center coordinates of the area position and the coverage area of the area position. The specific process of step S12212 may flexibly change along with the representation form of the area position. In one example, the process of step S12212 is: based on the center of gravity coordinates of the segmentation result in the feature map, respectively determining the center of gravity coordinates of the target object in each feature map in the convolution result according to a resolution proportional relation between the feature map where the segmentation result and the remaining feature maps in the convolution result; and taking the center of gravity coordinates as the center, in each feature image, determining the area capable of fully covering the target object, and taking coordinates of vertices of the area as corresponding position information of the target object in the convolution result. Because there is a resolution difference between feature maps in the convolution result, there may also be resolution difference between the areas covering the target object in the feature maps in the convolution result. In one example, there is a proportional relationship between the determined areas covering the target object in the different feature maps, and the proportional relationship is consistent with the resolution proportional relationship between the features. For example, in one example, if the convolution result may include two feature maps A and B, the area covering the target object in feature map A is denoted as area A, and the area covering the target object in feature map B is denoted as area B, where the resolution of feature map A is twice of the resolution of feature map B, the area of area A is twice of the area of area B.

Based on the position information obtained in step S1221, the positioning result is obtained by means of step S1222. The embodiments above indicate that the position information may exist in multiple difference representation forms, and as the representation form of the position information is different, the specific implementation process of step S1222 may also be different. In one possible implementation mode, step S1222 includes: respectively performing cropping processing on the feature map at each resolution in the convolution result according to the position information to obtain the positioning result. In one example, the position information may be a set of coordinates of vertices of the area covering the target object in feature map in the convolution result. Based on the coordinate set, each feature map in the convolution result is cropped, the area covering the target object in each feature map is reversed as a new feature map, and a set of the new feature maps is the positioning result.

According to a combination of the embodiments above in any form, the positioning result is obtained. This process may effectively perform rough positioning on the target object in the feature map at each resolution in the convolution result. Based on the rough positioning, the original convolution result is processed as the positioning result. Because most of image information not including the target object is removed from the feature map at each resolution in the positioning result, the storage consumption in the image processing process is greatly reduced, the calculation speed is accelerated, and the efficiency and speed of image processing are improved. Moreover, because the ratio of information of the target object in the position result is larger, the effect of performing target object segmentation based on the positioning result is better than the effect of performing target object segmentation directly using the image to be processed, so that the precision of image processing is improved.

After the positioning result is obtained, segmentation of the target object is implemented based on the positioning result. The specific implementation form of segmentation is not limited, and may be flexibly selected according to actual conditions. In one possible implementation mode, a certain feature map is selected from the positioning result, and then further segmentation processing is performed to obtain the target object. In another possible implementation mode, a feature map having more target object information may be restored from the positioning result, and then further segmentation processing is performed on the feature map to obtain the target object.

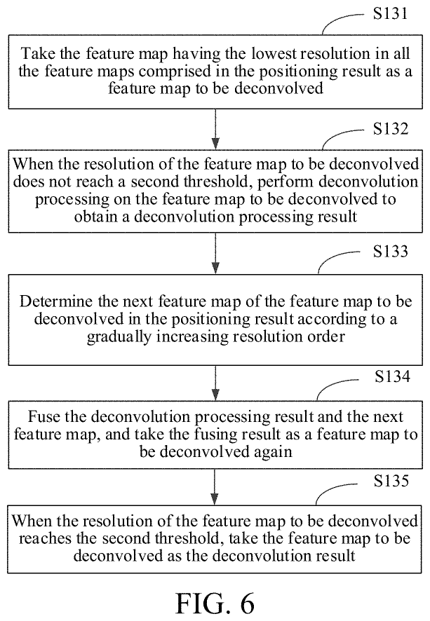

It can be seen from the steps above that in one possible implementation mode, the process of implementing target object segmentation using the positioning result may be implemented by steps S13 and S14. That is, step-by-step deconvolution processing is first performed on the positioning result to obtain the deconvolution result including more target object information, and then segmentation processing is performed based on the deconvolution result to obtain the target object. The step-by-step deconvolution process is considered as a reverse operation process of the step-by-step convolution process, and therefore, the implementation process also has a plurality of possible implementation forms as step S11. FIG. 6 is a flowchart illustrating an image processing method according to one embodiment of the present disclosure. As shown in FIG. 6, in one possible implementation mode, step S13 includes the following steps.

At step S131, the feature map having the lowest resolution in all the feature maps included in the positioning result is taken as a feature map to be deconvolved.

At step S132, when the resolution of the feature map to be deconvolved does not reach a second threshold, deconvolution processing is performed on the feature map to be deconvolved to obtain a deconvolution processing result.

At step S133, the next feature map of the feature map to be deconvolved in the positioning result is determined according to a gradually increasing resolution order.

At step S134, the deconvolution processing result and the next feature map are fused, and the fusing result is taken as a feature map to be deconvolved again.

At step S135, when the resolution of the feature map to be deconvolved reaches the second threshold, the feature map to be deconvolved is taken as the deconvolution result.

In the steps above, the deconvolution processing result is a processing result obtained by performing deconvolution processing on the feature map to be deconvolved, and the next feature map is a feature map obtained from the positioning result. That is, in the positioning result, a feature satisfying a condition that the resolution is greater than that of the current deconvolution feature map by one level may be taken as the next feature map to be fused with the deconvolution processing result. Therefore, the process of step-by-step deconvolution processing may be performing deconvolution processing from a feature map having the lowest resolution in the positioning result to obtain a feature map of which the resolution is increased by one level, and at this time, the feature map obtained by increasing the resolution by one level is taken as the deconvolution processing result. Because the positioning result also has a feature map having the same resolution as the deconvolution processing result, and both feature maps include valid information of the target object, the two feature images are fused. The fused feature map includes all the valid information of the target object included in the two feature maps, and therefore, the fused feature map is taken as a new feature map to be deconvolved again, the feature map to be deconvolved is subjected to deconvolution processing, and the processing result is fused with a feature map having the corresponding resolution in the positioning result again, until the resolution of the fused feature map reaches the second threshold, and the deconvolution processing ends. At this time, the obtained final fusing result includes all the valid information of the target object included in each feature map in the positioning result, and therefore may be taken as the deconvolution result for subsequent target object segmentation. In the embodiments of the present disclosure, the second threshold is flexibly decided according to the original resolution of the image to be processed, and the specific value is not limited herein.

In the process above, the deconvolution result is obtained by performing step-by-step deconvolution processing on the positioning result, and is used for final target object segmentation. Therefore, because there is a basis for positioning the target object, the obtained final result may effectively include global information of the target object, and has high accuracy. Moreover, there is no need to segment the image to be processed, but image processing is performed as a whole, and therefore, the processing process also has higher resolution. Moreover, it can be seen from the process above that in one image processing process, the segmentation of the target object is implemented based on the positioning result of the target object, there is no need to separately implement target object positioning and target object segmentation through two independent processes, and therefore, the storage, consumption and calculation amount of data are greatly reduced, the speed and efficiency of image processing are improved, and the consumption in time and space is reduced. Moreover, based on the step-by-step deconvolution process, valid information included in the feature maps at each resolution is reserved in the finally obtained deconvolution result, and because the deconvolution result is used for final image segmentation, the precision of the finally obtained result is greatly improved.

After the deconvolution result is obtained, segmentation processing is performed on the deconvolution result, and the obtained result is taken as the target object segmented from the image to be processed. The process for performing segmentation processing on the deconvolution result is consistent with the process for performing segmentation processing on the convolution result, there are only difference in the objects to be segmented, and therefore, the process in the embodiments above is referred to, and details are not described herein again.

In one possible implementation mode, the image processing method of the embodiments of the present disclosure is implemented by means of a neural network. It can be seen from the process that the image processing method of the embodiments of the present disclosure mainly includes two segmentation processes, where the first segmentation is rough segmentation on the image to be processed, and the second segmentation is segmentation with higher precision based on the positioning result of the rough segmentation. Therefore, the second segmentation and the first segmentation are implemented by one neural network and share a set of parameters. Therefore, the two segmentations may be seen as two sub-neural networks under one neural network. Therefore, in one possible implementation mode, the neural network includes a first segmentation sub-network and a second segmentation sub-network, where the first segmentation sub-network is configured to perform step-by-step convolution processing and segmentation processing on the image to be processed, and the second segmentation sub-network is configured to perform step-by-step deconvolution processing and segmentation processing on the positioning result. The specific network structure used by the neural network is not limited. In one example, V-Net and 3D-U-Net mentioned in the embodiments above both may serve as specific implementation modes of the neural network. Any neural network capable of implementing the functions of the first segmentation sub-network and the second segmentation sub-network may be the implementation mode of the neural network.

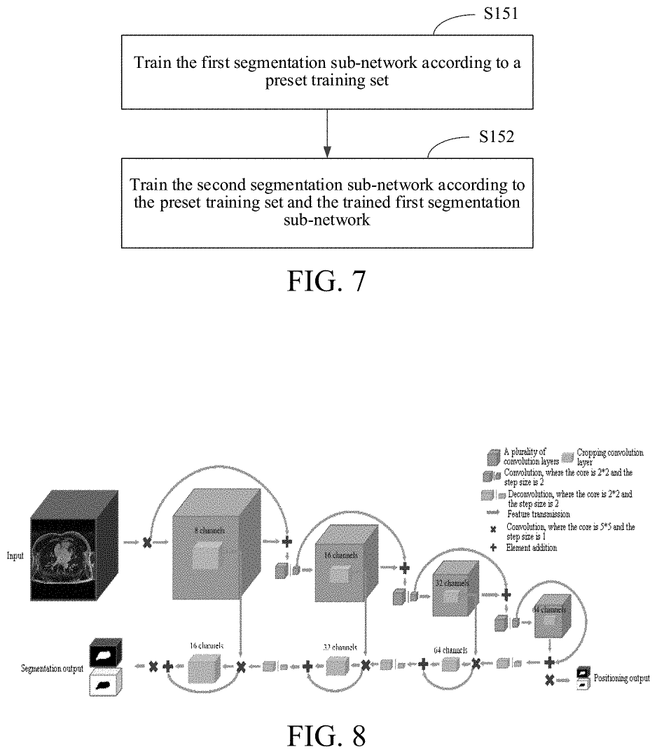

FIG. 7 is a flowchart illustrating an image processing method according to embodiments of the present disclosure. In one possible implementation mode, as shown in FIG. 7, the method of the embodiments of the present disclosure further includes a training process for the neural network, which is denoted as step S15, where step S15 includes the following steps.

At step S151, the first segmentation sub-network is trained according to a preset training set.

At step S152, the second segmentation sub-network is trained according to the preset training set and the trained first segmentation sub-network.

The preset training set may be a plurality of image sets obtained by dividing a sample image after preprocessing such as manual cropping. In the plurality of image sets obtained by division, adjacent two image sets may include a part of same images. For example, taking medical images as an example, a plurality of samples are collected from a hospital, a plurality of sample images in one sample may be images of a certain organ of the human body collected continuously, and a three-dimensional structure of the organ is obtained through the plurality of sample images. Division may be performed along one direction, a first image set includes the first to thirtieth image frames, the second image set includes the sixteenth to the forty-fifth image frames . . . , so that 15 images frames are the same between every adjacent two image sets. Through this overlapping division mode, the precision of cutting is improved.

As shown in FIG. 7, in the training process for the neural network, first a preset training set is taken as input to train the first segmentation sub-network, according to the output result of the first segmentation sub-network, positioning processing is performed on images in the training set, and the training set subjected to the positioning processing is taken as training data for the second segmentation sub-network and is input to the second segmentation sub-network for training. Through the process above, the trained first segmentation sub-network and second segmentation sub-network are finally obtained.

In the training process, a function used for determining a network loss of the neural network is not specifically limited. In one example, the network loss of the neural network may be determined through a dice loss function. In one example, the network loss of the neural network may be determined through a cross entropy function. In one example, the network loss of the neural network may also be determined by other available loss functions. The loss functions used for the first segmentation sub-network and the second segmentation sub-network may be the same, or different, which is not limited herein.

Based on the embodiments above, in one example, the complete training process for a neural network is: inputting a preset training set to a network model of the first segmentation sub-network, the preset training set includes a plurality of to-be-segmented images and masks corresponding to the to-be-segmented images, calculating a loss between data output after the images pass through the network model of the first segmentation sub-network and the corresponding masks through any loss function, and then updating a network model parameter of the first segmentation sub-network through a backpropagation algorithm, until the first segmentation sub-network model is converged, representing that the training for the first segmentation sub-network model is completed. After the training for the first segmentation sub-network model is completed, the preset training set is input to the trained first segmentation sub-network model again to obtain a plurality of segmentation results. Based on the plurality of segmentation results, positioning processing is performed on the feature maps under different resolution in the first segmentation sub-network, the positioned and cropped feature map and the masks of the corresponding positions are input to the network model of the second segmentation sub-network for training, a loss between data output after the images subjected to the positioning processing pass through the network model of the second segmentation sub-network and the corresponding masks is calculated through any loss function, then a network model parameter of the second segmentation sub-network is updated through a backpropagation algorithm, the network model parameters of the first segmentation sub-network and the second segmentation sub-network are updated alternately until the whole network model is converged, and the training for the neural network is completed.

It can be seen from the embodiments above that although the neural network in the present disclosure includes two sub-neural neural networks, in the training process, only one set of training set data is need for complete the training. The two sub-neural networks share the same set of parameters, and more storage space is saved. Because the trained two sub-neural networks share the same set of parameters, when the neural network is applied to the image processing method, the input image to be processed directly passes through the two sub-neural networks in sequence to obtain the output result, rather than separately inputting to the two sub-neural networks to respectively obtain output results and then performing calculation. Therefore, the image processing method provided in the present disclosure has a faster processing speed, and lower space consumption and time consumption.

In one possible implementation mode, the method of the embodiments of the present disclosure, before step S11, further includes: adjusting the image to be processed to preset resolution. The implementation method for adjusting the image to be processed to preset resolution is not specifically limited. In one example, the image to be processed is adjusted to preset resolution by using a central cropping and expansion method. The specific resolution value of the present resolution is likewise not limited, and is flexibly set according to actual conditions.

Based on this step, when the image processing method of the embodiments of the present disclosure is implemented through the neural network, the training images included in the preset training set may also be unified to the preset resolution and then be used for training of the neural network.

Accordingly, in one possible implementation mode, the method of the embodiments of the present disclosure further includes: restoring the segmented target object to a space having the size as the image to be processed to obtain the final segmentation result. Because the resolution of the image to be processed may be adjusted before step S11, the obtained segmentation result actually may be segmented content of the image subjected to resolution adjustment, and therefore, the segmentation result is restored to the space having the same size as the image to be processed to obtain the segmentation result based on the original image to be processed. The space having the same size as the image to be processed is not limited and is decided according to image properties of the image to be processed, which is not limited herein. In one example, the image to be processed may be a three-dimensional image, and therefore, the space having the same size as the image to be processed is a three-dimensional space.

In one possible implementation mode, before step S11, the method further includes: preprocessing the image to be processed. The preprocessing process is not limited, and any processing mode capable of improving the segmentation precision may be taken as a process included in preprocessing. In one example, the preprocessing on the image to be processed may include performing brightness value equalization on the image to be processed.

By using images to be processed under the same resolution as input to perform image processing, the processing efficiency for subsequently performing convolution processing, segmentation processing, and step-by-step deconvolution processing on the images to be processed is improved, and the time of the entire image processing process is shortened. By preprocessing the image to be processed, the degree of accuracy of image segmentation is improved, and thus the precision of the image processing result is improved.

Application Scenario Example

Heart disease is one of the diseases with the highest fatality rate, for example, atrial fibrillation one of the most common heart rate disorders at present, with a probability of 2% in the general population, and a higher incidence and certain fatality rate exist in the elder population, which severely threatens the human health. Moreover, precise segmentation on the atrium is the key of understanding and analyzing atrial fibrillation, and is generally used for assisting in formulating a surgical ablation therapeutic plan targeting the atrial fibrillation. Moreover, segmentation on other cavities of the heart is equally significant to therapeutic and surgical planning for heart diseases of other types. However, methods for segmenting heart cavities in a medical image still have defects such as poor accuracy and low calculation efficiency. Although there are some methods achieving relatively high accuracy, there are still some actual problems, such as the lack of three-dimensional information, not enough smoothness in segmentation result, lack of global information, low calculation efficiency, or the need for performing segmentation training in two split networks, or certain degrees of redundancy in both time and space.

Therefore, a segmentation method having high precision, high efficiency, and low time-space consumption may greatly reduce the workload of doctors, improve the quality of heart segmentation, and thus enhance the therapeutic effect for heart-related diseases.

FIG. 8 is a schematic diagram of one application example of the present disclosure. As shown in FIG. 8, the embodiments of the present disclosure provide an image processing method, which is implemented based on a set of trained neural networks. It can be seen from FIG. 8 that the specific training process for the set of neural networks is:

first processing preset training data, where the preset training data includes a plurality of input images and corresponding masks, and unifying the resolution of the plurality of input images to the same magnitude by using a central cropping and expansion method, where the unified resolution in the present example is 576×576×96.

After the resolution of the plurality of input images is unified, the input images are used to train the first segmentation sub-network, and the specific training process is:

performing convolution processing on the input images for multiple times by using an encoder structure in a neural network similar to a V-Net- or 3D-U-Net-based three-dimensional full convolutional neural network, where the convolution processing process in the present example includes convolution, pooling, batch norm, and PRelu, after multiple times of convolution processing, the input of each convolution processing is the result obtained in the last convolution processing, four times of convolution processing are executed in the present example, and therefore, feature maps having the resolution of 576×576×96, 288×288×48, 144×144×24, and 72×72×12 are respectively generated, and channels for inputting the images are increased to 128 from 8;

after the four feature maps are obtained, regarding the feature map having the lowest resolution, which is the feature map of 72×72×12 in the present example, enabling the feature map to pass through a softmax layer to obtain two probability outputs having the resolution of 72×72×12, where the two probability outputs respectively represent the probabilities whether pixel related positions are a target cavity, and are taken as the output result of the first segmentation sub-network; using a dice loss, cross entropy or other loss functions to calculate a loss between the output result and the mask that is directly down-sampled to 72×72×12, and based on the calculated loss, updating a network parameter of the first segmentation sub-network by using a backpropagation algorithm until the network model of the first segmentation sub-network is converged, which represents that the training for the first segmentation sub-network is completed.

After the training for the first segmentation sub-network is completed, the plurality of input images having unified resolution passes through the trained first segmentation sub-network to obtain four feature maps having the resolution of 576×576×96, 288×288×48, 144×144×24, and 72×72×12, and two probability outputs having resolution of 72×72×12. According to the probability outputs of the low resolution, a rough segmentation result for the heart cavity is obtained by using maximum value comparison, where the resolution is 72×72×12. Based on the rough segmentation result, the coordinates of the center of gravity of the heart cavity are calculated, and areas which have fixed sizes and are capable of fully covering the target cavity are cropped from the four feature maps having the resolution of 576×576×96, 288×288×48, 144×144×24, and 72×72×12 by taking the coordinates of the center of gravity as the center. In one example, an area having a size of 30×20×12 is cropped from the feature map of 72×72×12, an area having a size of 60×40×24 is cropped from the feature map of 144×144×24, an area having a size of 120×80×48 is cropped from the feature map of 288×288×48, and an area having a size of 240×160×96 is cropped from the feature map of 576×576×96.

After the four cropped area images are obtained, the second segmentation sub-network is trained by using the area images, and the specific training process is:

restoring the area images to resolution of 240×160×96 step by step by using step-by-step deconvolution, where the specific process is: performing deconvolution processing on the area having the size of 30×20×12 cropped from the feature map of 72×72×12 to obtain the feature map having the resolution of 60×40×24, fusing this feature map with the area having the size of 60×40×24 cropped from the feature map of 144×144×24 to obtain the fused feature map having the resolution of 60×40×24, then performing deconvolution processing on this feature map to obtain the feature map having the resolution of 120×80×48, fusing this feature map with the area having the size of 120×80×48 cropped from the feature map of 288×288×48 to obtain the fused feature map having the resolution of 120×80×48, performing deconvolution processing on the fused feature map to obtain the feature map having the resolution of 240×160×96, and fusing this feature map with the area having the size of 240×160×96 cropped from the feature map of 576×576×96 to obtain the final image after the step-by-step deconvolution processing, where the final image includes local and global information of the heart cavity; enabling the final image to pass through the softmax layer to obtain two probability outputs having the resolution of 576×576×96, where the two probability outputs respectively represent the probabilities whether pixel related positions are a target cavity, and are taken as the output result of the second segmentation sub-network; and then using a dice loss, cross entropy or other loss functions to calculate a loss between the output result and the mask, and based on the calculated loss, updating a network parameter of the second segmentation sub-network by using a backpropagation algorithm until the network model of the second segmentation sub-network is converged, which represents that the training for the second segmentation sub-network is completed.

Through the steps above, a trained neural network for heart cavity segmentation is obtained, positioning and segmentation on the heart cavity are complemented simultaneously in the same neural network, and the result is directly obtained after the image passes through the network. Therefore, the heart cavity segmentation process based on the trained neural network is specifically:

first adjusting the resolution of the to-be-segmented image to be subjected to heat cavity segmentation to a preset size, which is 576×576×96 in the present example of the neural network by using a central cropping and expansion method, and then inputting the to-be-segmented image data to the trained neural network, where the to-be-segmented image goes through a process similar to the training process in the trained neural network, i.e., first generating feature maps of four resolutions by means of convolution processing, then obtaining a rough segmentation result, cropping the feature maps of the four resolutions based on the rough segmentation result, performing deconvolution processing on the cropping result to obtain the deconvolution result, performing segmentation processing on the deconvolution result to obtain the segmentation result of the target cavity, outputting the segmentation result as the output result of the neural network, and mapping the output segmentation result to the same dimension size as the input to-be-segmented image, i.e., obtaining the final heart cavity segmentation result.

Using the image processing method of the present disclosure, the heart cavity may be positioned and segmented using one three-dimensional network. Positioning and segmentation share the same set of parameters. Positioning and segmentation of the heart cavity are unified to the same network, and therefore, the segmentation result is directly obtained from the input by one step. A higher speed is achieved, more storage space is saved, and moreover, a smoother three-dimensional model segmentation surface is obtained.

It should be noted that the image processing method of the embodiments of the present disclosure is not limited to application in heart cavity image processing, and may be applied to any image processing, which is not limited by the present disclosure.

It may be understood that the foregoing method embodiments mentioned in the present disclosure may be combined with each other to obtain a combined embodiment without departing from the principle and the logic. Details are not described in the present disclosure due to space limitation.

A person skilled in the art can understand that, in the foregoing methods of the specific implementations, the order in which the steps are written does not imply a strict execution order which constitutes any limitation to the implementation process, and the specific order of executing the steps should be determined by functions and possible internal logics thereof.

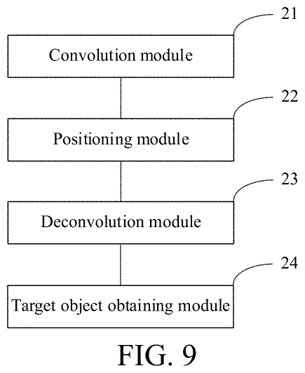

FIG. 9 is a block diagram illustrating an image processing apparatus according to an embodiment of the present disclosure. The image processing apparatus may be a terminal device, a terminal, other processing device, or the like. The terminal device may be a User Equipment (UE), a mobile device, a user terminal, a terminal, a cellular phone, a cordless telephone, a Personal Digital Assistant (PDA), a handheld device, a computer device, a vehicle-mounted device, a wearable device, or the like.

In some possible implementation modes, the image processing apparatus may be implemented by invoking, by a processor, computer readable instructions stored in a memory.

As shown in FIG. 9, the image processing apparatus includes: a convolution module 21, configured to perform step-by-step convolution processing on an image to be processed to obtain a convolution result; a positioning module 22, configured to obtain a positioning result through positioning processing according to the convolution result; a deconvolution module 23, configured to perform step-by-step deconvolution processing on the positioning result to obtain a deconvolution result; and a target object obtaining module 24, configured to perform segmentation processing on the deconvolution result to segment a target object from the image to be processed.

In one possible implementation mode, the convolution module is configured to: perform step-by-step convolution processing on the image to be processed to obtain at least one feature map having gradually decreasing resolution as the convolution result.

In one possible implementation mode, the convolution module is further configured to: perform convolution processing on the image to be processed, where an obtained feature map serves as a feature map to be convolved; the resolution of the feature map to be convolved does not reach a first threshold, perform convolution processing on the feature map to be convolved and take the obtained result as a feature map to be convolved again; and when the resolution of the feature map to be convolved reaches the first threshold, take all the feature maps having the gradually decreasing resolution as the convolution result.

In one possible implementation mode, the positioning module includes: a segmentation sub-module, configured to perform segmentation processing according to the convolution result to obtain a segmentation result; and a positioning sub-module, configured to perform positioning processing on the convolution result according to the segmentation result to obtain the positioning result.

In one possible implementation mode, the segmentation sub-module is configured to: perform segmentation processing on the feature map having the lowest resolution in the convolution result to obtain the segmentation result.

In one possible implementation mode, the positioning sub-module is configured to: determine corresponding position information of the target object in the convolution result according to the segmentation result; and perform positioning processing on the convolution result according to the position information to obtain the positioning result.

In one possible implementation mode, the positioning sub-module is further configured to: read a coordinate position of the segmentation result; and taking the coordinate position as an area center, respectively determine, in the convolution result, an area position capable of fully covering the target object in the feature map at each resolution as the corresponding position information of the target object in the convolution result.

In one possible implementation mode, the positioning sub-module is further configured to: respectively perform cropping processing on the feature map at each resolution in the convolution result according to the position information to obtain the positioning result.

In one possible implementation mode, the deconvolution module is configured to: take the feature map having the lowest resolution in all the feature maps included in the positioning result as a feature map to be deconvolved; when the resolution of the feature map to be deconvolved does not reach a second threshold, perform deconvolution processing on the feature map to be deconvolved to obtain a deconvolution processing result; determine the next feature map of the feature map to be deconvolved in the positioning result according to a gradually increasing resolution order; fuse the deconvolution processing result and the next feature map, and take the fusing result as a feature map to be deconvolved again; and when the resolution of the feature map to be deconvolved reaches the second threshold, take the feature map to be deconvolved as the deconvolution result.

In one possible implementation mode, the segmentation processing includes: performing softmax regression on an object to be segmented to obtain a regression result; and performing maximum value comparison on the regression result to complete the segmentation processing on the object to be segmented.

In one possible implementation mode, the apparatus is implemented by a neural network, and the neural network includes a first segmentation sub-network and a second segmentation sub-network, where the first segmentation sub-network is configured to perform step-by-step convolution processing and segmentation processing on the image to be processed, and the second segmentation sub-network is configured to perform step-by-step deconvolution processing and segmentation processing on the positioning result.

In one possible implementation mode, the apparatus further includes a training module, configured to: train the first segmentation sub-network according to a preset training set; and train the second segmentation sub-network according to the preset training set and the trained first segmentation sub-network.

In one possible implementation mode, before the convolution module, the apparatus further includes a resolution adjusting module, configured to: adjust the image to be processed to preset resolution.

The embodiments of the present disclosure further provide a computer readable storage medium having computer program instructions stored thereon, where the foregoing method is implemented when the computer program instructions are executed by a processor. The computer readable storage medium may be a non-volatile computer readable storage medium.

The embodiments of the present disclosure further provide an electronic device, including: a processor; and a memory configured to store processor-executable instructions, where the processor is configured to execute the foregoing methods.

The electronic device may be provided as a terminal, a server, or devices in other forms.

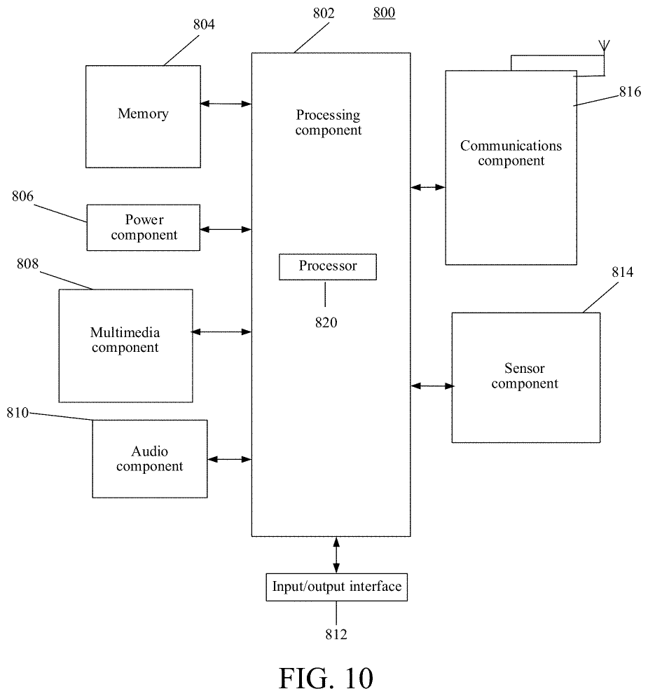

FIG. 10 is a block diagram of an electronic device 800 according to embodiments of the present disclosure. For example, the electronic device 800 may be a terminal such as a mobile phone, a computer, a digital broadcast terminal, a message transceiver device, a game console, a tablet device, a medical device, a fitness device, or a personal digital assistant.

Referring to FIG. 10, the electronic device 800 may include one or more of the following components: a processing component 802, a memory 804, a power supply component 806, a multimedia component 808, an audio component 810, an Input/Output (I/O) interface 812, a sensor component 814, and a communications component 816.

The processing component 802 usually controls the overall operation of the electronic device 800, such as operations associated with display, telephone call, data communication, a camera operation, or a recording operation. The processing component 802 may include one or more processors 820 to execute instructions, to complete all or some of the steps of the foregoing method. In addition, the processing component 802 may include one or more modules, for convenience of interaction between the processing component 802 and other components. For example, the processing component 802 may include a multimedia module, for convenience of interaction between the multimedia component 808 and the processing component 802.

The memory 804 is configured to store data of various types to support an operation on the electronic device 800. For example, the data includes instructions, contact data, phone book data, a message, an image, or a video of any application program or method that is operated on the electronic device 800. The memory 804 may be implemented by any type of volatile or non-volatile storage device or a combination thereof, such as a Static Random Access Memory (SRAM), an Electrically Erasable Programmable Read-Only Memory (EEPROM), an Erasable Programmable Read-Only Memory (EPROM), a Programmable Read-Only Memory (PROM), a Read-Only Memory (ROM), a magnetic memory, a flash memory, a magnetic disk, or an optical disc.

The power supply component 806 supplies power to various components of the electronic device 800. The power supply component 806 may include a power management system, one or more power supplies, and other components associated with power generation, management, and allocation for the electronic device 800.

The multimedia component 808 includes a screen that provides an output interface and is between the electronic device 800 and a user. In some embodiments, the screen may include a Liquid Crystal Display (LCD) and a Touch Panel (TP). If the screen includes the touch panel, the screen may be implemented as a touchscreen, to receive an input signal from the user. The touch panel includes one or more touch sensors to sense a touch, a slide, and a gesture on the touch panel. The touch sensor may not only sense a boundary of a touch operation or a slide operation, but also detect duration and pressure related to the touch operation or the slide operation. In some embodiments, the multimedia component 808 includes a front-facing camera and/or a rear-facing camera. When the electronic device 800 is in an operation mode, for example, a photographing mode or a video mode, the front-facing camera and/or the rear-facing camera may receive external multimedia data. Each front-facing camera or rear-facing camera may be a fixed optical lens system that has a focal length and an optical zoom capability.

The audio component 810 is configured to output and/or input an audio signal. For example, the audio component 810 includes one microphone (MIC). When the electronic device 800 is in an operation mode, such as a call mode, a recording mode, or a voice recognition mode, the microphone is configured to receive an external audio signal. The received audio signal may be further stored in the memory 804 or sent by using the communications component 816. In some embodiments, the audio component 810 further includes a speaker, configured to output an audio signal.

The I/O interface 812 provides an interface between the processing component 802 and a peripheral interface module, and the peripheral interface module may be a keyboard, a click wheel, a button, or the like. These buttons may include, but are not limited to, a home button, a volume button, a startup button, and a lock button.

The sensor component 814 includes one or more sensors, and is configured to provide status evaluation in various aspects for the electronic device 800. For example, the sensor component 814 may detect an on/off state of the electronic device 800 and relative positioning of components, and the components are, for example, a display and a keypad of the electronic device 800. The sensor component 814 may also detect a location change of the electronic device 800 or a component of the electronic device 800, existence or nonexistence of contact between the user and the electronic device 800, an orientation or acceleration/deceleration of the electronic device 800, and a temperature change of the electronic device 800. The sensor component 814 may include a proximity sensor, configured to detect existence of a nearby object when there is no physical contact. The sensor component 814 may further include an optical sensor, such as a CMOS or CCD image sensor, configured for use in imaging application. In some embodiments, the sensor component 814 may further include an acceleration sensor, a gyro sensor, a magnetic sensor, a pressure sensor, or a temperature sensor.

The communications component 816 is configured for wired or wireless communication between the electronic device 800 and other devices. The electronic device 800 may be connected to a communication-standard-based wireless network, such as Wi-Fi, 2G or 3G, or a combination thereof. In an exemplary embodiment, the communications component 816 receives a broadcast signal or broadcast-related information from an external broadcast management system through a broadcast channel. In an exemplary embodiment, the communications component 816 further includes a Near Field Communication (NFC) module, to facilitate short-range communication. For example, the NFC module is implemented based on a Radio Frequency Identification (RFID) technology, an Infrared Data Association (IrDA) technology, an Ultra Wideband (UWB) technology, a Bluetooth (BT) technology, and other technologies.

In an exemplary embodiment, the electronic device 800 may be implemented by one or more of an Application Specific Integrated Circuit (ASIC), a Digital Signal Processor (DSP), a Digital Signal Processing Device (DSPD), a Programmable Logic Device (PLD), a Field Programmable Gate Array (FPGA), a controller, a microcontroller, a microprocessor, or other electronic components, and is configured to perform the foregoing method.

In an exemplary embodiment, a non-volatile computer readable storage medium, for example, the memory 804 including computer program instructions, is further provided. The computer program instructions may be executed by the processor 820 of the electronic device 800 to complete the foregoing method.

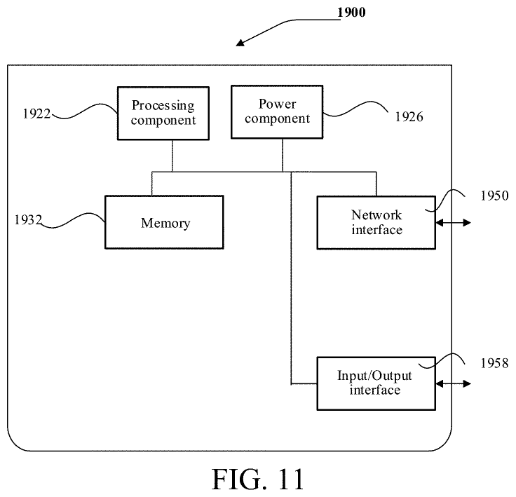

FIG. 11 is a block diagram of an electronic device 1900 according to embodiments of the present disclosure. For example, the electronic device 1900 may be provided as a server. Referring to FIG. 11, the electronic device 1900 includes a processing component 1922 that further includes one or more processors; and a memory resource represented by a memory 1932, configured to store instructions, for example, an application program, that may be executed by the processing component 1922. The application program stored in the memory 1932 may include one or more modules each corresponding to a set of instructions. In addition, the processing component 1922 is configured to execute the instructions to perform the foregoing method.

The electronic device 1900 may further include: a power supply component 1926, configured to perform power management of the electronic device 1900; a wired or wireless network interface 1950, configured to connect the electronic device 1900 to a network; and an Input/Output (I/O) interface 1958. The electronic device 1900 may operate an operating system stored in the memory 1932, such as Windows Server™, Mac OS X™, Unix™, Linux™, or FreeBSD™.

In an exemplary embodiment, a non-volatile computer readable storage medium, for example, the memory 1932 including computer program instructions, is further provided. The computer program instructions may be executed by the processing component 1922 of the electronic device 1900 to complete the foregoing method.

The present disclosure may be a system, a method, and/or a computer program product. The computer program product may include a computer readable storage medium, and computer readable program instructions that are used by the processor to implement various aspects of the present disclosure are loaded on the computer readable storage medium.

The computer readable storage medium may be a tangible device that can maintain and store instructions used by an instruction execution device. The computer-readable storage medium may be, but is not limited to, an electronic storage device, a magnetic storage device, an optical storage device, an electromagnetic storage device, a semiconductor storage device, or any suitable combination of the above ones. More specific examples (a non-exhaustive list) of the computer readable storage medium include a portable computer disk, a hard disk, a Random Access Memory (RAM), a Read-Only Memory (ROM), an Erasable Programmable Read-Only Memory (EPROM or flash memory), a Static Random Access Memory (SRAM), a portable Compact Disc Read-Only Memory (CD-ROM), a Digital Versatile Disk (DVD), a memory stick, a floppy disk, a mechanical coding device such as a punched card storing instructions or a protrusion structure in a groove, and any appropriate combination thereof. The computer readable storage medium used here is not interpreted as an instantaneous signal such as a radio wave or another freely propagated electromagnetic wave, an electromagnetic wave propagated by a waveguide or another transmission medium (for example, an optical pulse transmitted by an optical fiber cable), or an electrical signal transmitted by a wire.

The computer readable program instructions described here may be downloaded from a computer readable storage medium to each computing/processing device, or downloaded to an external computer or an external storage device via a network, such as the Internet, a local area network, a wide area network, and/or a wireless network. The network may include a copper transmission cable, optical fiber transmission, wireless transmission, a router, a firewall, a switch, a gateway computer, and/or an edge server. A network adapter or a network interface in each computing/processing device receives the computer readable program instructions from the network, and forwards the computer readable program instructions, so that the computer readable program instructions are stored in a computer readable storage medium in each computing/processing device.

Computer program instructions for carrying out operations of the present disclosure may be assembler instructions, Instruction-Set-Architecture (ISA) instructions, machine instructions, machine dependent instructions, microcode, firmware instructions, state-setting data, or either source code or object code written in any combination of one or more programming languages, including an object oriented programming language such as Smalltalk, C++ or the like, and conventional procedural programming languages, such as the “C” programming language or similar programming languages. The program readable program instructions may be completely executed on a user computer, partially executed on a user computer, executed as an independent software package, executed partially on a user computer and partially on a remote computer, or completely executed on a remote computer or a server. In the case of a remote computer, the remote computer may be connected to a user computer via any kind of network, including a Local Area Network (LAN) or a Wide Area Network (WAN), or may be connected to an external computer (for example, connected via the Internet with the aid of an Internet service provider). In some embodiments, an electronic circuit such as a programmable logic circuit, a Field Programmable Gate Array (FPGA), or a Programmable Logic Array (PLA) is personalized by using status information of the computer readable program instructions, and the electronic circuit may execute the computer readable program instructions to implement various aspects of the present disclosure.

Various aspects of the present disclosure are described here with reference to the flowcharts and/or block diagrams of the methods, apparatuses (systems), and computer program products according to the embodiments of the present disclosure. It should be understood that each block in the flowcharts and/or block diagrams and a combination of the blocks in the flowcharts and/or block diagrams may be implemented by using the computer readable program instructions.

These computer readable program instructions may be provided for a general-purpose computer, a dedicated computer, or a processor of another programmable data processing apparatus to generate a machine, so that when the instructions are executed by the computer or the processor of the another programmable data processing apparatus, an apparatus for implementing a specified function/action in one or more blocks in the flowcharts and/or block diagrams is generated. These computer readable program instructions may also be stored in a computer readable storage medium, and these instructions may instruct a computer, a programmable data processing apparatus, and/or another device to work in a specific manner. Therefore, the computer readable storage medium storing the instructions includes an artifact, and the artifact includes instructions for implementing a specified function/action in one or more blocks in the flowcharts and/or block diagrams.

The computer readable program instructions may be loaded onto a computer, another programmable data processing apparatus, or another device, so that a series of operations and steps are executed on the computer, the another programmable apparatus, or the another device, thereby generating computer-implemented processes. Therefore, the instructions executed on the computer, another programmable apparatus, or another device implement a specified function/action in one or more blocks in the flowcharts and/or block diagrams.

The flowcharts and block diagrams in the accompanying drawings show possible architectures, functions, and operations of the systems, methods, and computer program products in the embodiments of the present disclosure. In this regard, each block in the flowcharts or block diagrams may represent a module, a program segment, or a part of instruction, and the module, the program segment, or the part of instruction includes one or more executable instructions for implementing a specified logical function. In some alternative implementations, functions marked in the block may also occur in an order different from that marked in the accompanying drawings. For example, two consecutive blocks are actually executed substantially in parallel, or are sometimes executed in a reverse order, depending on the involved functions. It should also be noted that each block in the block diagrams and/or flowcharts and a combination of blocks in the block diagrams and/or flowcharts may be implemented by using a dedicated hardware-based system that executes a specified function or action, or may be implemented by using a combination of dedicated hardware and a computer instruction.

Different embodiments in the present application may be mutually combined without violating logic. The different embodiments emphasize different aspects, and for a part not described in detail, reference may be made to descriptions of other embodiments.

The embodiments of the present disclosure are described above. The foregoing descriptions are exemplary but not exhaustive, and are not limited to the disclosed embodiments. For a person of ordinary skill in the art, many modifications and variations are all obvious without departing from the scope and spirit of the described embodiments. The terms used in the specification are intended to best explain the principles of the embodiments, practical applications, or technical improvements to the technologies in the market, or to enable others of ordinary skill in the art to understand the embodiments disclosed in the specification.

1. An image processing method, comprising:
performing step-by-step convolution processing on an image to be processed to obtain a convolution result;
obtaining a positioning result through positioning processing according to the convolution result;
performing step-by-step deconvolution processing on the positioning result to obtain a deconvolution result; and
performing segmentation processing on the deconvolution result to segment a target object from the image to be processed.

2. The method according to claim 1, wherein performing step-by-step convolution processing on the image to be processed to obtain the convolution result comprises:
performing step-by-step convolution processing on the image to be processed to obtain at least one feature map having gradually decreasing resolution as the convolution result.

3. The method according to claim 2, wherein performing step-by-step convolution processing on the image to be processed to obtain the at least one feature map having the gradually decreasing resolution as the convolution result comprises:
performing convolution processing on the image to be processed, wherein the obtained feature map serves as a feature map to be convolved;
when the resolution of the feature map to be convolved does not reach a first threshold, performing convolution processing on the feature map to be convolved and taking the obtained result as a feature map to be convolved again; and
when the resolution of the feature map to be convolved reaches the first threshold, taking all the feature maps having the gradually decreasing resolution as the convolution result.

4. The method according to claim 1, wherein obtaining the positioning result through positioning processing according to the convolution result comprises:
performing segmentation processing according to the convolution result to obtain a segmentation result; and
performing positioning processing on the convolution result according to the segmentation result to obtain the positioning result.

5. The method according to claim 4, wherein performing segmentation processing according to the convolution result to obtain the segmentation result comprises:
performing segmentation processing on the feature map having the lowest resolution in the convolution result to obtain the segmentation result.

6. The method according to claim 4, wherein performing positioning processing on the convolution result according to the segmentation result to obtain the positioning result comprises:
determining corresponding position information of the target object in the convolution result according to the segmentation result; and
performing positioning processing on the convolution result according to the position information to obtain the positioning result.

7. The method according to claim 6, wherein determining the corresponding position information of the target object in the convolution result according to the segmentation result comprises:
reading a coordinate position of the segmentation result; and
taking the coordinate position as an area center, and respectively determining, in the convolution result, an area position capable of fully covering the target object in the feature map at each resolution as the corresponding position information of the target object in the convolution result.

8. The method according to claim 6, wherein performing positioning processing on the convolution result according to the position information to obtain the positioning result comprises:
respectively performing cropping processing on the feature map at each resolution in the convolution result according to the position information to obtain the positioning result.

9. The method according to claim 1, wherein performing step-by-step deconvolution processing on the positioning result to obtain the deconvolution result comprises:
taking the feature map having the lowest resolution in all the feature maps comprised in the positioning result as a feature map to be deconvolved;
when the resolution of the feature map to be deconvolved does not reach a second threshold, performing deconvolution processing on the feature map to be deconvolved to obtain a deconvolution processing result;
determining a next feature map of the feature map to be deconvolved in the positioning result according to a gradually increasing resolution order;
fusing the deconvolution processing result and the next feature map, and taking the fusing result as a feature map to be deconvolved again; and
when the resolution of the feature map to be deconvolved reaches the second threshold, taking the feature map to be deconvolved as the deconvolution result.

10. The method according to claim 1, wherein the segmentation processing comprises:
performing softmax regression on an object to be segmented to obtain a regression result; and
performing maximum value comparison on the regression result to complete the segmentation processing on the object to be segmented.

11. The method according to claim 1,
wherein the method is implemented by a neural network, and the neural network comprises a first segmentation sub-network and a second segmentation sub-network,
wherein the first segmentation sub-network is configured to perform step-by-step convolution processing and segmentation processing on the image to be processed, and the second segmentation sub-network is configured to perform step-by-step deconvolution processing and segmentation processing on the positioning result.

12. The method according to claim 11, wherein a training process for the neural network comprises:
training the first segmentation sub-network according to a preset training set; and
training the second segmentation sub-network according to the preset training set and the trained first segmentation sub-network.

13. The method according to claim 1, before performing step-by-step convolution processing on the image to be processed to obtain the convolution result, further comprising: adjusting the image to be processed to preset resolution.

14. The method according to claim 1, wherein the image to be processed is a three-dimensional medical image.

15. An image processing apparatus, comprising:
a processor; and
a memory configured to store processor-executable instructions,
wherein the processor is configured to invoke the instructions stored in the memory, so as to:
perform step-by-step convolution processing on an image to be processed to obtain a convolution result;
obtain a positioning result through positioning processing according to the convolution result;
perform step-by-step deconvolution processing on the positioning result to obtain a deconvolution result; and
perform segmentation processing on the deconvolution result to segment a target object from the image to be processed.

16. The apparatus according to claim 15, wherein performing step-by-step convolution processing on the image to be processed to obtain the convolution result comprises:
performing step-by-step convolution processing on the image to be processed to obtain at least one feature map having gradually decreasing resolution as the convolution result.

17. The apparatus according to claim 16, wherein performing step-by-step convolution processing on the image to be processed to obtain the at least one feature map having the gradually decreasing resolution as the convolution result comprises:
performing convolution processing on the image to be processed, wherein the obtained feature map serves as a feature map to be convolved;
when the resolution of the feature map to be convolved does not reach a first threshold, performing convolution processing on the feature map to be convolved and taking the obtained result as a feature map to be convolved again; and
when the resolution of the feature map to be convolved reaches the first threshold, taking all the feature maps having the gradually decreasing resolution as the convolution result.

18. The apparatus according to claim 15, wherein obtaining the positioning result through positioning processing according to the convolution result comprises:
performing segmentation processing according to the convolution result to obtain a segmentation result; and
performing positioning processing on the convolution result according to the segmentation result to obtain the positioning result.

19. The apparatus according to claim 18, wherein performing segmentation processing according to the convolution result to obtain the segmentation result comprises:
performing segmentation processing on the feature map having the lowest resolution in the convolution result to obtain the segmentation result.

20. A non-transitory computer-readable storage medium having computer program instructions stored thereon, wherein when the computer program instructions are executed by a processor, the processor is caused to perform the operations of:
performing step-by-step convolution processing on an image to be processed to obtain a convolution result;
obtaining a positioning result through positioning processing according to the convolution result;
performing step-by-step deconvolution processing on the positioning result to obtain a deconvolution result; and
performing segmentation processing on the deconvolution result to segment a target object from the image to be processed.
